(19) United States
(12) Patent Application Publication
(10) Pub. No.: US 2024/0385269 A1
(43) Pub. Date: Nov. 21, 2024

(54) T1rho DISPERSION CHARACTERIZATION BY MAGNETIC RESONANCE FINGERPRINTING

(71) Applicant: The Cleveland Clinic Foundation, Cleveland, OH (US)

(72) Inventors: Xiaojuan Li, Cleveland, OH (US); Brendan Eck, Cleveland, OH (US); Jeehun Kim, Cleveland, OH (US)

(21) Appl. No.: 18/667,174

(22) Filed: May 17, 2024

Related U.S. Application Data
(60) Provisional application No. 63/467,792, filed on May 19, 2023.

(51) Int. Cl. G01R 33/50; A61B 5/00; G01R 33/56

(52) U.S. Cl. CPC G01R 33/50; A61B 5/004; G01R 33/5608

(57) ABSTRACT

Magnetic resonance fingerprinting (MRF) is used to quantify T_{1ρ} dispersion across spin-lock frequencies and enables simultaneous mapping of T₁, T₂, and T_{1ρ} dispersion. T_{1ρ} at a specific FSL may be determined by retrospectively computation based on the results of the T_{1ρ} dispersion MRF dictionary and pattern matching results. The T_{1ρ} dispersion characteristic may be used to identify and track tissue parameters (such as osteoarthritis and muscle degeneration) before being expressed in other measurable manners.

### CROSS-REFERENCE TO RELATED APPLICATIONS

This application claims the benefit of U.S. provisional application Ser. No. 63/467,792 filed May 19, 2023, the entire contents of which are incorporated by reference.

### STATEMENT OF GOVERNMENT-SPONSORED RESEARCH

This invention was made with government support under AG070321 and AR007505 awarded by the National Institutes of Health. The government has certain rights in the invention.

### BACKGROUND

The value of medical imaging research has grown significantly in recent decades. Techniques such as magnetic resonance fingerprinting (MRF), functional magnetic resonance imaging (fMRI), magnetic resonance spectroscopy (MRS), diffusion tensor imaging (DTI), and positron emission tomography (PET) provide a non-invasive method of exploring the structure and function of tissues within the body. These advanced imaging techniques have played a crucial role in advancing many fundamental areas of medical science, including the study of cancer, multiple sclerosis, soft tissue injuries, joint or spinal injuries, Parkinson's disease, Alzheimer's disease, and other conditions.

In particular, MRF is characterized by a pseudo-randomized acquisition strategy, pattern matching, and tissue property visualization. MRF is a flexible, rapid imaging technique that can allow simultaneous quantification of multiple tissue properties. Generally MRF involves generating an MRI pulse sequence by varying various MR acquisition parameters. Data acquired from these pulse sequencies represent “fingerprints” that are different for different tissues/tissue properties. These fingerprints (e.g., from each pixel or voxel of the acquired data) can then be pattern matched to a dictionary of known fingerprints to identify the type of tissue and/or its properties.

### BRIEF SUMMARY OF THE INVENTION

According to a first example of the present disclosure, a magnetic resonance fingerprinting (MRF) method comprises: applying an MRF pulse sequence to tissue of a subject; acquiring magnetic resonance (MR) signal data as a result of the application of the MRF pulse sequence; comparing the MR signal data to a predefined MRF dictionary, the MRF dictionary comprising a model of T_{1ρ} dispersion; and determining a property of the tissue from the T_{1ρ} dispersion model based on a result of the comparison.

In various embodiments of the above disclosure, T_{1ρ} dispersion is modeled according to:

$${T}_{1 ρ} ( ω ) = \frac{{( {q}^{2} D )}^{2} + {ω}^{2}}{{γ}^{2} {g}^{2} D} = {{m}^{2} ( {q}^{2} D )}^{2} + {m}^{2} {ω}^{2}$$

where T_{1ρ}(0)=m²(q²D)²=T₂ and T_{1ρ}(ω)=T₂+(mω)² where γ is a hydrogen gyromagnetic ratio, D is a self-diffusion coefficient, q is a spatial frequency of a local magnetic field variation, g is a mean local magnetic gradient strength, ω is frequency, and m is a mediation coefficient that mediates a strength of a T_{1ρ} dispersion effect and represents a combination of γ, D, and g; wherein T_{1ρ} dispersion is modeled according to: T_{1ρ}(ω)=T₂+mω where ω is frequency, and m is a mediation coefficient that mediates a strength of a T_{1ρ} dispersion effect and represents a combination of a hydrogen gyromagnetic ratio, a self-diffusion coefficient, and a mean local magnetic gradient strength; the determined tissue property relates to tissue degeneration; the method further comprises: identifying an osteoarthritis or muscle degeneration condition in the subject based on the identified property; the method further comprises: applying a plurality MRF pulse sequencies, and tracking a change in T_{1ρ} dispersion of the tissue of the subject over the plurality of applied MRF sequences; the method further comprises: applying a plurality MRF pulse sequencies, and tracking a change in the determined tissue property over the plurality of applied MRF sequences; the MRF pulse sequence includes a single fixed spin-lock frequency; the method further comprises: determining a T_{1ρ} dispersion by retrospectively determining T_{1ρ} at a plurality of spin-lock frequencies based on the model of the MRF dictionary; the MRF dictionary comprises fingerprints of at least T₁, T₂, and m; and/or the MR signal data is compared to the predefined MRF dictionary with a machine learning system trained to identify MR properties of the MRF dictionary based on input MR signal data.

### BRIEF DESCRIPTION OF THE DRAWINGS

FIG. 1 illustrates an example method for characterizing T_{1ρ} dispersion in accordance with the present disclosure.

FIG. 2 illustrates T₁, T₂, and T_{1ρ} dispersion from MRF as compared to a ground truth reference.

FIG. 3 illustrates T_{1ρ} relaxation time maps retrospectively generated at spin-lock frequencies (FSL) of FSL=50, 100, 200, and 500 Hz from MRF as compared to ground truth gencrated maps.

FIG. 4 illustrates measured T_{1ρ} and R_{1ρ} in ROIs of the retrospectively MRF-derived maps as compared to the ground truth dispersion curve.

FIG. 5 illustrates MRF estimated T₁, T₂, and T_{1ρ} dispersion maps as well as MAPSS T₂ and T_{1ρ} dispersion maps for an agarose and glucose phantom.

### DETAILED DESCRIPTION OF THE DRAWING

Considering the above, the present disclosure relates to a data acquisition and reconstruction framework, and more particularly, a framework to enable T_{1ρ} dispersion magnetic resonance fingerprinting (MRF) data acquisition and reconstruction.

In magnetic resonance imaging, an MRI pulse sequence is a particular setting of radiofrequency excitation pulses and field gradient waveforms timed in a manner that yields a particular image appearance. A multiparametric MRI is a combination of two or more sequences, and/or includes other specialized MRI configurations such as spectroscopy. A pulse sequence is generally defined by multiple parameters including time to echo (TE), time to repetition (TR), flip angle, field of view and matrix size, inversion pulses, spoiler gradients, echo train length (ETL), special acquisition of k-space, post contrast imagine, and diffusion weighting. These pulse sequences can be broadly grouped into either a spin echo sequence, an inversion recovery sequence, a gradient echo sequence, a diffusion weighted sequence, saturation recovery sequences, echo-planar pulse sequences, and spiral pulse sequences. Multiple sequences are used evaluate a tissue, and the combination of sequences is referred to as an MRI protocol.

Commonly, tissues are characterized by relaxation times T₁ and T₂. T₁ is the time constant that determines the rate at which excited protons return to equilibrium. T₁ is a measure of the time taken for spinning protons to realign with an external magnetic field. T₂ is the time constant that determines the rate at which the excited protons reach equilibrium and is a measure of the time taken for spinning protons to lose phase coherence among the nuclei spinning perpendicular to the main magnetic field.

The most common MRI sequences are T₁-weighted and T₂-weighted scans. T₁-weighted scans use short TE and TR times, while T₂-weighted scans use longer TE and TR times. In T₁-weighted images, the contrast and brightness are determined by T₁ properties of the tissues, while in T₂-weighted images, the contrast and brightness are determined by T₂ properties.

A less common MRI sequence includes T_{1ρ}. T_{1ρ} is a potential biomarker of musculoskeletal diseases such as osteoarthritis and muscle degeneration. T_{1ρ} has elements of both T₁ and T₂ weighting, and is dependent on the T₁ and T₂ of the tissue, but can additionally select for different properties within the tissue. The relaxation time of T_{1ρ} is a tissue property that can be probed in order to assess tissue composition (e.g., glycosaminoglycan content in cartilage and fiber type proportion in skeletal muscle). T_{1ρ} characterizes longitudinal relaxation during the application of a “spin-locking” radiofrequency excitation magnetic (B₁) pulse. T_{1ρ} is influenced by interactions of large biomolecules and water, and is dependent on the spin-lock frequency (FSL) of the applied B₁ field. And the dispersion of T_{1ρ} across FSL values is an additional magnetic property beyond single-frequency T_{1ρ} that is sensitive to chemical exchange and diffusion or diffusive exchange.

T_{1ρ} relaxation can be quantified via multiple spin-lock magnetization preparation pulses at a given FSL with varied spin-lock times (TSL) to acquire T_{1ρ} weighted images. Typically, curve-fitting of T_{1ρ} weighted images is performed using an exponential decay equation. By quantifying T_{1ρ} at multiple FSL values, the T_{1ρ} dispersion of a tissue can be characterized.

T_{1ρ} dispersion represents a relatively new approach to MRI tissue characterization. Advantageously, T_{1ρ} dispersion is thought to allow for higher sensitivity and specificity when analyzing some particular tissues and tissue properties compared to traditional T₁ and T₂. With T_{1ρ} dispersion, the T_{1ρ} signal across FSL from tissues is amplified which increases the sensitivity and therefore ability to detect macromolecular components in tissue. Indeed, disease processes may be detected based on large molecules directly rather than indirectly through their effects on water, as in T₁ and T₂ imaging.

Additionally, T_{1ρ} dispersion can be monitored for a single patient over a period of time to track changes a patient's tissue properties. Thus, dispersion may allow for carlier detection of tissue degeneration, musculoskeletal changes, and other tissue-related changes that could be used to assess and diagnose various diseases. These MRF properties may be related to particular tissue properties via normative databases (e.g., collections of related properties among large populations), or via pattern recognition techniques such as those utilizing machine learning systems.

Monitoring T_{1ρ} and T_{1ρ} dispersion using traditional MR techniques can be prohibitively time consuming as they generally would require multiple scans at different FSL. They can also require prohibitively high energy deposition rates at relatively high FSL values. These factors can be barriers in acquiring T_{1ρ} information. However, the use of magnetic resonance fingerprinting (MRF) methods can be utilized to reduce scan times and high energy deposition. Utilizing MRF in this way allows for the measurement of multiple tissue properties in a single acquisition because each tissue type has a unique signal response to a given input sequences which depends on its physical, chemical, and biological properties. Further T_{1ρ} can be determined from a scan at a single, fixed FSL, and T_{1ρ} dispersion MRF can yield T_{1ρ} maps at a single FSL retrospectively.

The embodiments described herein serve to improve conventional MRF by providing a framework to enable T_{1ρ} dispersion MRF data acquisition and reconstruction. According to the present disclosure, T₁, T₂, and T_{1ρ} can be simultaneously mapped, and MRF can be used to quantify T_{1ρ} at a single spin-lock frequency. According to the techniques of the present disclosure, maps of T₁, T₂, and T_{1ρ} dispersion can be generated. Those maps can then be used to retrospectively create T_{1ρ} relaxation time maps without having to measure T_{1ρ} at each FSL.

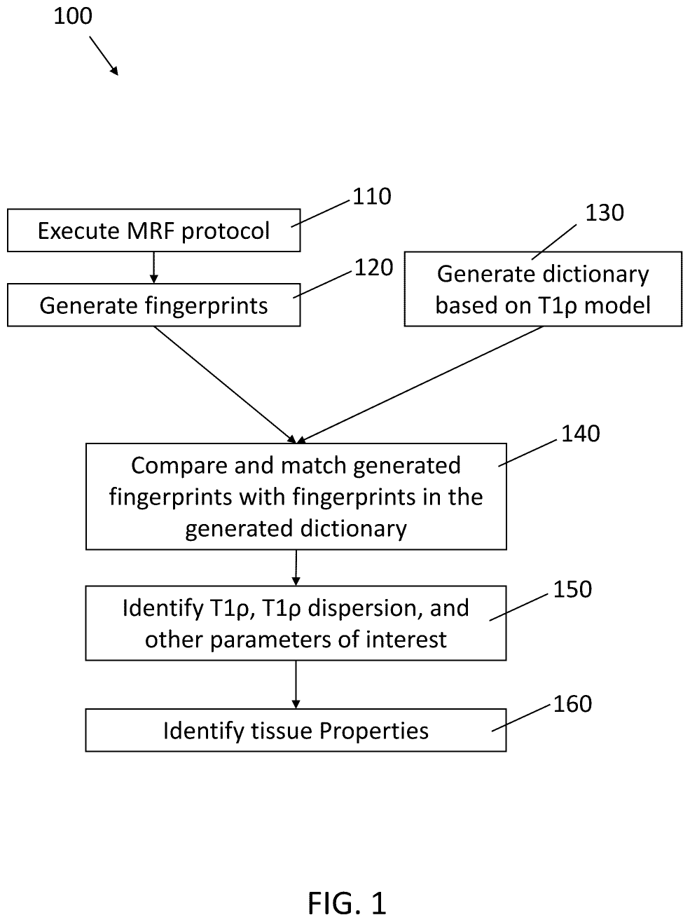

Turning to the figures, FIG. 1 illustrates a flowchart of an example method 100 according to the present disclosure. As with other implementations of MRF, the method begins by executing an MRF protocol 110, or a series of pulse sequences, and acquiring associated MR signal data or fingerprints 120 in response. Here, the executed MRF protocol scan parameters include FSL, flip angle, repetition time, and echo time, which may be varied or fixed. In one example embodiment, the FSL is fixed, and the T_{1ρ} mapping can be made retrospectively for other (non-scanned) FSL.

As noted above, the fingerprints from the application of the MRF protocol 110 are then compared to and matched with a collection of simulated fingerprints in a gencrated dictionary 130 of expected signal patterns. In some embodiments, this comparison may be performed by pattern matching techniques, such as with machine learning systems. In some embodiments, the comparison may be performed with look-up tables or the like. If an exact match is not found in such a table, a closest match may be selected or parameters may be determined by interpolation or similar techniques between two or more closest matches.

The generated dictionary 130 models T_{1ρ} dispersive effects across FSL, and can relate T_{1ρ}, FSL, hydrogen gyromagnetic ratio, self-diffusion coefficient, spatial frequency of a local magnetic field variation, and related mean gradient strength. The T_{1ρ} dispersion MRF method uses variable FSL, variable flip angle, fixed repetition time, and fixed echo time in this implementation, but is not restricted to fixed repetition time or fixed echo time. Depending on the embodiment, various models may be used to determine/model the value of T_{1ρ}. According to one example, the dictionary models T_{1ρ} dispersion by the following:

$${T}_{1 ρ} ( ω ) = \frac{{( {q}^{2} D )}^{2} + {ω}^{2}}{{γ}^{2} {g}^{2} D} = {{m}^{2} ( {q}^{2} D )}^{2} + {m}^{2} {ω}^{2} \qquad ( Equation 1 )$$

$$where$$

$${T}_{1 ρ} ( 0 ) = {{m}^{2} ( {q}^{2} D )}^{2} = {T}_{2} and {T}_{1 ρ} ( ω ) = {T}_{2} + {( m ω )}^{2} \qquad ( Equation 2 )$$

Equations (1) and (2) above relate T_{1ρ}, FSL(ω), hydrogen gyromagnetic ratio (γ), self-diffusion coefficient (D), spatial frequency of a local magnetic field variation (q), related mean gradient strength (g), and a mediation coefficient (m) that mediates the strength of a T_{1ρ}dispersion effect and represents a combination of γ, D, and g. A larger value of m represents a greater dispersion, and a zero value represents no dispersion. The quantities g²D and q²D are related to T₂ by the following:

$${T}_{2} = \frac{{( {q}^{2} D )}^{2}}{{γ}^{2} {g}^{2} D} \qquad ( Equation 3 )$$

$${m}^{2} = \frac{1}{{γ}^{2} {g}^{2} D} \qquad ( Equation 4 )$$

at FSL=0 and T_{1ρ}=T₂.

In other embodiments, T_{1ρ} may be modeled with a linear dispersion model such as:

$${T}_{1 ρ} ( ω ) = {T}_{2} + m ω \qquad ( Equation 5 )$$

After the MRF dictionary models T_{1ρ} dispersion using the above formulae, dictionary generation proceeds as before, but with T_{1ρ} relaxation being dependent on FSL. T₁, T₂, T_{1ρ} dispersion, and with m being directly mapped instead of T₁, T₂, and T_{1ρ}. A single FSL still leads to T_{1ρ} mapping, and MRF without T_{1ρ} mapping results in m=0. Data acquisition is carried out as in existing T_{1ρ} MRF methods, but with varied FSL.

The dictionary is generated to contain three parameters that characterize the relevant magnetic characteristics of a tissue—namely, T₁, T₂, and m. After acquisition of T_{1ρ} dispersion MRF data, a 3D spatial total variation-regularized, temporal low rank reconstruction can be employed to suppress noise and undersampling artifacts and obtain tissue property maps.

The fingerprints are then compared to and matched with 140 the collection of fingerprints in the generated dictionary. As a result, T₁, T₂, T_{1ρ} dispersion, and m are all directly mapped instead of just T₁, T₂, and T_{1ρ}. Once fingerprints are matched 140, the properties of interest (such as T_{1ρ}) may be identified 150. As noted above, this MRF property may be related to tissue properties based on normative databases or like techniques, and may be monitored over a period of time to identify and/or track disease progression.

The above technique has been verified using at least two phantoms. In one example, a digital cylindrical phantom with known ground truth T₁, T₂, and T_{1ρ} dispersion values was used for in silico implementation of the T_{1ρ} dispersion MRF sequence. Particularly, a 500 frame MRF sequence was used with the acceleration factor R=60 and a matrix size of 192×192×16.

In another example, an agarose gel phantom with glucose addition was used to assess T_{1ρ} dispersion using T_{1ρ} dispersion MRF as compared to a reference method. During validation, the MRF sequence utilized a 15-channel Tx/Rx knee coil, while the reference method was a T₂/T_{1ρ} magnetization-prepared angle-modulated partitioned k-space spoiled gradient echo snapshots (MAPSS) at a matrix size of 192×192×16 with T1ρ quantifications at FSL=50, 100, 200, and 500 Hz. Four echoes were acquired for each FSL, with TSL=0, 10, 30, and 70 ms. A total scan time was 10 minutes. The aforementioned T_{1ρ} dispersion model was fitted to MAPSS T_{1ρ} values for comparison with MRF. T_{1ρ} MRF used the same sequence settings as in simulation but with a matrix size 96×96×16 and a 2.5 minute scan time.

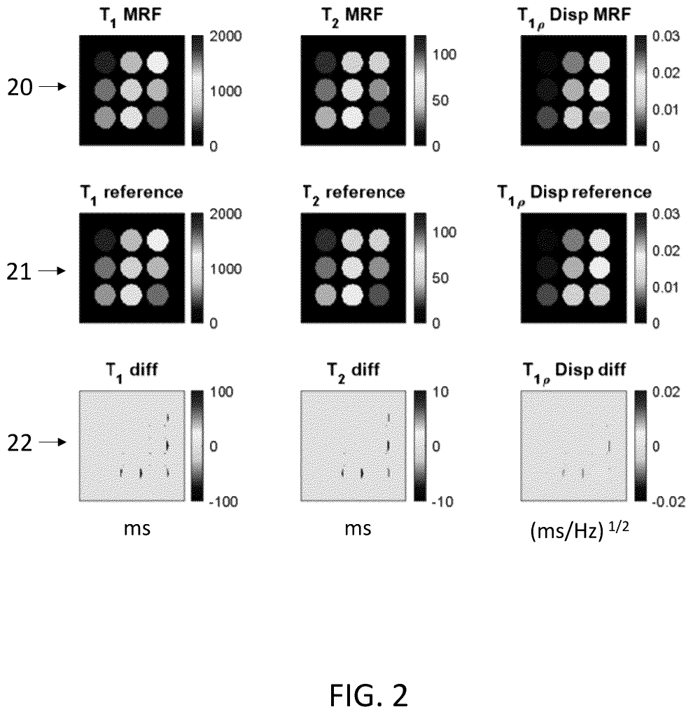

FIGS. 2-5 illustrate the results of these simulations. In particular, FIG. 2 illustrates T₁, T₂, and T_{1ρ} dispersion from MRF 20 as compared to a ground truth reference 21. As illustrated, by comparing the respective maps of each of T₁, T₂, and T_{1ρ} generated by MRF 20 to those generated by the ground truth reference 21, the difference between T_{1ρ} dispersion MRF and T_{1ρ} dispersion reference is minimal compared to the differences between T₁ and T₂ and their respective ground truth references. This difference is visually illustrated in map sequence 22.

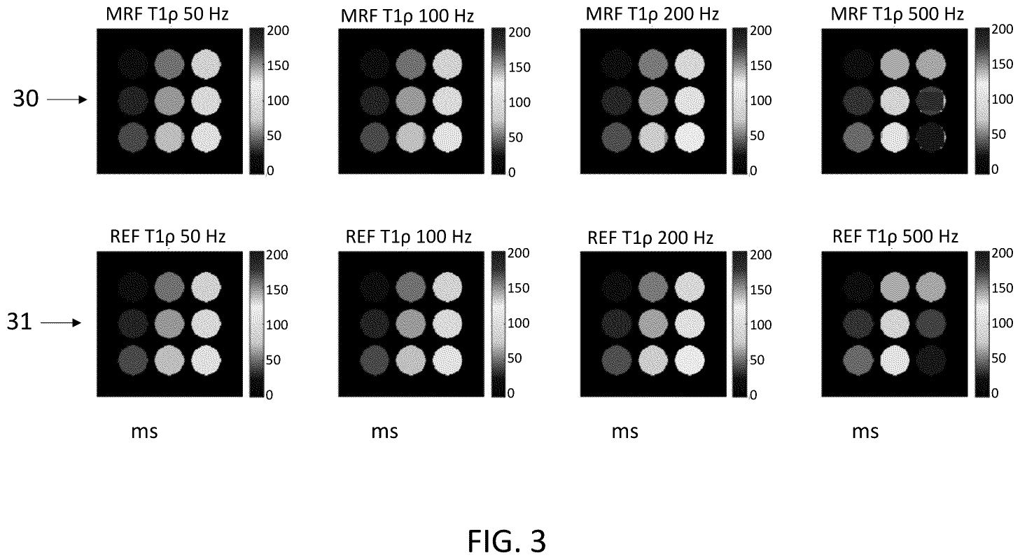

FIG. 3 illustrates T_{1ρ} dispersion maps 30 retrospectively generated at FSL=50, 100, 200, and 500 Hz from MRF as compared to ground truth generated maps 31. Again, comparison between the MRF maps 30 and the maps generated via the reference technique are extremely similar at each FSL.

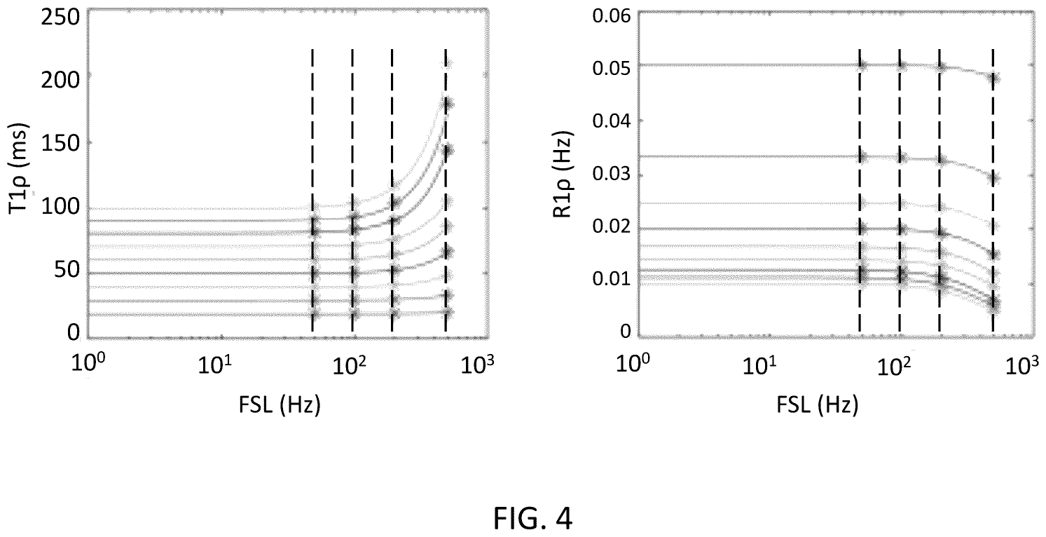

Similarly, FIG. 4 illustrates the measured T_{1ρ} and R₁ₚ, where R_{1ρ}=1/T_{1ρ}, in regions of interest (ROIs) of the retrospectively MRF-derived maps illustrated in FIG. 3 as compared to the ground truth dispersion curve (solid lines). The FSL frequencies of each of the maps of FIS. 3 (50, 100, 200, and 500 Hz) are marked by the vertical dashed lines on the graphs of FIG. 4, and the ground truth is indicated by the solid lines.

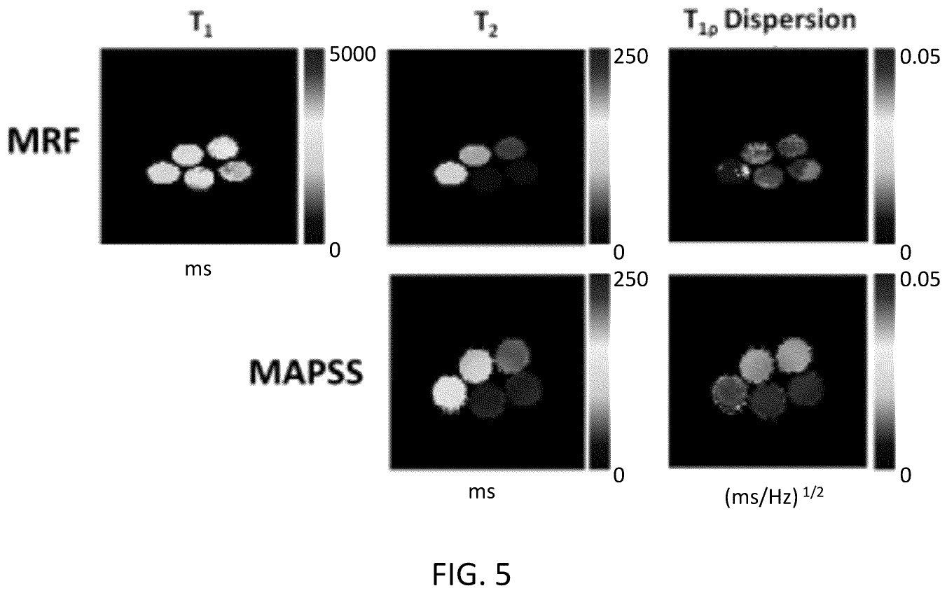

FIG. 5 further illustrates differences observed in T₂ and T_{1ρ} dispersion maps for the agarose and glucose phantom for the MRF technique and for the MAPSS technique. As illustrated, minimal differences are observed between MRF and MAPSS particularly with respect to in T_{1ρ} dispersion.

Simply, FIGS. 2-5 illustrate strong agreement between the MRF technique described herein and ground truth references and the MAPSS technique for all of T₁, T₂, and T_{1ρ}. To the extent errors were present, they tended to be greater for high dispersion values for both simulation and physical phantoms. Herein, while the various maps illustrated in FIGS. 2-5 are shown in grayscale/black-and-white, they may be represented in color.

Considering this, the feasibility of direct characterization of T_{1ρ} dispersion by MRF is possible according to the technique described herein. This is supported by simulations showing strong agreement between ground truth and MRF estimated parameters. With phantoms, there is generally good agreement between T_{1ρ} dispersion maps derived from conventional T_{1ρ} mapping and T_{1ρ} dispersion generated by the MRF technique described herein.

Moreover, T_{1ρ} dispersion via MRF can significantly reduce the required acquisition time and radiofrequency pulse energy deposition or amplifier requirements as compared to conventional T_{1ρ} dispersion collection methods due to the acquisition of MR signal data at a single fixed FSL rather than different spin-lock frequencies. The MRF T_{1ρ} dispersion technique herein further has the benefit of providing T₁ and T₂ maps in addition to the T_{1ρ} dispersion model parameters. Thus, T_{1ρ} dispersion acquisition may be achieved with a greater range of applications and system hardware.

Any aspect of the above disclosure may be implemented by a process of an MRI system, or by a processor of a separate computing device(s). It is further noted than any aspect may be executed locally (e.g., at the site of the MRI system or of the MRI imaging), or remotely. For example, MR data acquisition may be performed locally to the MRI system at a hospital or like clinical location, while other aspects of the present disclosure are performed at a remote central processing location and at a different time than the data acquisition. Still further, it is noted that the various aspects of the present disclosure may be distributed across any number of processors and/or computing systems.

While various features are presented above, it should be understood that the features may be used singly or in any combination thereof. Further, it should be understood that variations and modifications may occur to those skilled in the art to which the claimed examples pertain.

What is claimed is:

1. A magnetic resonance fingerprinting (MRF) method, comprising:
applying an MRF pulse sequence to tissue of a subject;
acquiring magnetic resonance (MR) signal data as a result of the application of the MRF pulse sequence;
comparing the MR signal data to a predefined MRF dictionary, the MRF dictionary comprising a model of T_{1ρ} dispersion; and
determining a property of the tissue from the T_{1ρ} dispersion model based on a result of the comparison.

2. The method according to claim 1, wherein T_{1ρ} dispersion is modeled according to:
$${T}_{1 ρ} ( ω ) = \frac{{( {q}^{2} D )}^{2} + {ω}^{2}}{{γ}^{2} {g}^{2} D} = {{m}^{2} ( {q}^{2} D )}^{2} + {m}^{2} {ω}^{2}$$
$$where$$
$${T}_{1 ρ} ( 0 ) = {{m}^{2} ( {q}^{2} D )}^{2} = {T}_{2} and {T}_{1 ρ} ( ω ) = {T}_{2} + {( m ω )}^{2}$$
where γ is a hydrogen gyromagnetic ratio, D is a self-diffusion coefficient, q is a spatial frequency of a local magnetic field variation, g is a mean local magnetic gradient strength, ω is frequency, and m is a mediation coefficient that mediates a strength of a T_{1ρ} dispersion effect and represents a combination of y, D, and g.

3. The method according to claim 1, wherein T_{1ρ} dispersion is modeled according to:
$${T}_{1 ρ} ( ω ) = {T}_{2} + m ω$$
where ω is frequency, and m is a mediation coefficient that mediates a strength of a T_{1ρ} dispersion effect and represents a combination of a hydrogen gyromagnetic ratio, a self-diffusion coefficient, and a mean local magnetic gradient strength.

4. The method according to claim 1, wherein the determined tissue property relates to tissue degeneration.

5. The method according to claim 1, further comprising:
identifying an osteoarthritis or muscle degeneration condition in the subject based on the identified property.

6. The method of claim 1, further comprising:
applying a plurality MRF pulse sequencies; and
tracking a change in T_{1ρ} dispersion of the tissue of the subject over the plurality of applied MRF sequences.

7. The method of claim 1, further comprising:
applying a plurality MRF pulse sequencies; and
tracking a change in the determined tissue property over the plurality of applied MRF sequences.

8. The method according to claim 1, wherein the MRF pulse sequence includes a single fixed spin-lock frequency.

9. The method according to claim 8, further comprising:
determining a T_{1ρ} dispersion by retrospectively determining T_{1ρ} at a plurality of spin-lock frequencies based on the model of the MRF dictionary.

10. The method according to claim 1, wherein the MRF dictionary comprises fingerprints of at least T₁, T₂, and m.

11. The method according to claim 1, wherein the MR signal data is compared to the predefined MRF dictionary with a machine learning system trained to identify MR properties of the MRF dictionary based on input MR signal data.
